(19) United States
(12) Patent Application Publication
(10) Pub. No.: US 2025/0122507 A1
(43) Pub. Date: Apr. 17, 2025

(54) DEPLETION OF FNDC5 REDUCES CANCER INDUCED MUSCLE LOSS/CACHEXIA

(71) Applicant: The Trustees of Indiana University, Bloomington, IN (US)

(72) Inventors: Lynda Bonewald, Indianapolis, IN (US); Fabrizio Pin, Indianapolis, IN (US); Andrea Bonetto, Indianapolis, IN (US); Anika Shimonty, Indianapolis, IN (US)

(21) Appl. No.: 18/694,650

(22) PCT Filed: Aug. 23, 2022
(86) PCT No.: PCT/US2022/041192

§ 371 Date: Mar. 22, 2024

Related U.S. Application Data
(60) Provisional application No. 63/247,954, filed on Sep. 24, 2021.

(51) Int. Cl. C12N 15/113; A61K 45/06; A61P 19/08; A61P 21/00; C07K 16/26; C07K 16/28; C12N 15/86

(52) U.S. Cl. CPC C12N 15/1138; A61K 45/06; A61P 19/08; A61P 21/00; C07K 16/26; C07K 16/28; C12N 15/1136; C12N 15/86; C12N 2310/14; C12N 2740/15043; C12N 2750/14143

(57) ABSTRACT

The present disclosure provides methods for preventing loss of muscle mass and function and bone loss in patients via gene therapy, administration of therapeutics, and combinations thereof. Methods to prevent loss of muscle mass and function in patients with cancer or any other condition that causes muscle wasting conditions such as sarcopenia, aging, immobilization, and any other cause of muscle loss, include inhibiting or depleting skeletal muscle of FNDC5/irisin. More particularly, the present disclosure provides antibody antagonists or other small molecules directed against FNDC5/irisin, for example, to reduce weakness in the patient and allow them to recover strength more quickly after eradication of the tumor.

### CROSS REFERENCE TO RELATED APPLICATIONS

This application is a U.S. National Phase Application of International PCT Application No. PCT/US2022/041192, filed Aug. 23, 2022, which claims priority to U.S. Provisional Application No. 63/247,954, filed Sep. 24, 2021. The contents of each application are incorporated herein by reference in their entireties.

### REFERENCE TO GOVERNMENT GRANTS

This invention was made with government support under AG039355 awarded by National Institutes of Health. The government has certain rights.

### FIELD OF THE INVENTION

The general field of the present disclosure is preventing loss of muscle mass and function and retention of bone mass in patients with cancer or any other condition that causes muscle wasting conditions such as sarcopenia, aging, immobilization, and any other cause of muscle loss. More particularly, the present disclosure provides gene therapy, administration of therapeutics, and combinations thereof, to prevent loss of muscle mass and function and loss of bone. Some method embodiments include administration of antibody antagonists or other small molecules directed against FNDC5 and/or irisin, for example, to reduce weakness in the patient and allow him/her to recover strength more quickly after eradication of the tumor.

### BACKGROUND

Cachexia is defined by abnormal loss of body weight and muscle mass that occurs secondary to chronic diseases, such as cancer. It is estimated that up to 80% of patients with advanced cancer will develop highly debilitating musculoskeletal dysfunction due to muscle loss and weakness during the course of their disease, dramatically impacting patient survival and leading to poorer outcomes. Several pathogenic mechanisms contribute to cachexia. Tumor-derived factors and elevated levels of pro-inflammatory cytokines are known to drive the imbalance between muscle protein synthesis and degradation rates as well as alterations in overall metabolism, that together promote muscle weakness and muscle atrophy. Unfortunately, no treatments are available for cachexia.

Fibronectin type III domain-containing protein 5 (FNDC5) is a single transmembrane protein whose mRNA is mainly expressed in skeletal muscles and in different organs, such as heart, kidney, brain, and pancreas. See Bostrom et al., “A PGC1-alpha-dependent myokine that drives brown-fat-like development of white fat and thermogenesis,” Nature. 2012; 481: pp. 463-8. It is proteolytically cleaved in a similar manner to PGC-1α, and secreted as the hormone “irisin”, suggesting that some of the beneficial effects of exercise could be mediated by this hormone. See. Wrann et al., “Exercise induces hippocampal BDNF through a PGC-1alpha/FNDC5 pathway,” Cell Metab. 2013; 18: pp. 649-59.

It has also been shown that FNDC5/irisin is associated with tumor growth in certain cancers. See for example Gaggini et al., “Increased FNDC5/Irisin expression in human hepatocellular carcinoma,” Peptides 88 (2017), pp. 62-66. However, the literature is confusing as irisin has been shown to have either beneficial or negative associations with tumor growth and metastasis. Further, because of its association muscle and muscle wasting, FNDC5 and/or irisin (hereinafter “FNDC5/irisin”) could represent a potential target for musculoskeletal disease in cancer patients and other conditions of skeletal muscle wasting and fatigue.

What is needed are novel treatments for musculoskeletal disease in cancer patients and to develop therapeutics aimed to prevent skeletal muscle wasting and fatigue in order to promote better outcomes in cachectic cancer patients. The present disclosure addresses this need.

### SUMMARY OF THE DISCLOSURE

The present disclosure is based on the discovery that FNDC5/irisin is an important mediator of skeletal muscle wasting and weakness and bone loss due to cancer and show that FNDC5/Irisin is an important target in the treatment and prevention of cancer cachexia.

The inventors have utilized a mouse model with a global deletion of FNDC5/irisin in order to demonstrate the potential therapeutic benefits in muscle wasting and fatigue and retention of bone mass. These animals were injected with a lung cancer cell line called Lewis Lung Carcinoma, LLC. Surprisingly, the mice lacking FNDC5/irisin did not lose any muscle mass (as shown in the preliminary data below). This dramatic effect has not been shown in any animal model or with any potential therapeutic. There are two potential explanations; one is that removing circulating irisin will prevent tumor-induced muscle cachexia, and the second is that FNDC5 may function independently of irisin in muscle, potentially as a receptor for tumor factors or tumor-derived cytokines. For example, FNDC5 may function as a receptor for tumor-or host-derived factor(s), which negatively regulate skeletal muscle mass and function or its cleavage product irisin may be responsible.

The findings shown herein provide evidence that FNDC5/irisin is an important mediator of skeletal muscle wasting and weakness and show that FNDC5/irisin is an important target in the treatment and prevention of cancer cachexia. Thus, neutralizing, blocking, reducing FNDC5/irisin through neutralizing antibody, small molecule inhibitors, shRNA, etc. will reduce or prevent muscle wasting and bone loss with cancer cachexia. Neutralizing antibodies are also provided to irisin in order to block or prevent cancer cachexia.

Thus, in some embodiments, FNDC5/irisin can be neutralized or inhibited by an agent where the agent comprises an adeno-associated virus (AAV) or lentovirus-containing an a short-hairpin RNA (shRNA) against FNDC5/irisin (shFNDC5/irisin). In some embodiments, the shFNDC5/irisin is commercially available and can be attached to or part of any vector known in the art including plasmids, viral vectors, bacteriophages, cosmids, and artificial chromosomes.

In other embodiments, the agent comprises a monoclonal antibody directed against the FNDC5/irisin. In yet other embodiments, the agent comprises a monoclonal antibody directed against FNDC5/irisin. In still other embodiments, the agent is an siRNA or antisense oligonucleotide that targets FNDC5/irisin.

In still other embodiments, the agent is an antagonist that binds to a FNDC/irisin-mediated receptor and prevents the binding of FNCS/irisin.

In embodiments of the current disclosure, methods are provided for treating or preventing muscle wasting and bone loss in cancer cachexia and other disorders. In particular embodiments, the methods involve providing treatments that inhibit or ablate FNDC5/irisin in a particular tumor.

In yet other embodiments, the methods of the current disclosure will provide means of preserving body wasting and muscle and bone mass during cancer cachexia and in other conditions associated with skeletal muscle derangements.

In still other embodiments, the methods of targeting FNDC5/irisin are used to prevent other conditions of muscle loss such as sarcopenia with aging, muscle and bone loss with immobilization, and other conditions of muscle loss or wasting.

The current invention provides pharmaceutical compositions comprising an agent that neutralizes or reduced FNDC5/irisin. The pharmaceutical compositions of the current invention can further comprise one or more pharmaceutically acceptable carriers, diluents or excipients. In any of the methods of the current invention, can further comprise the administration of one or more additional therapeutic agents.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1A-C shows the absence of FNDC5/irisin preserves body weight (BW) in LLC and MC38 hosts. Body weight changes (FIG. 1A), tumor-free final body weight (FBW; FIG. 1B) and tumor mass (FIG. 1C). Data are expressed as mean±SD. Significant differences: **P<0.01, ***P<0.001 versus WT; $P<0.05 versus LLC WT or MC38 WT.

FIG. 2A-C shows that absence of FNDC5/irisin preserves body composition. Carcass weight (FIG. 2A), lean mass (FIG. 2B) and fat mass (FIG. 2C) determined by DEXA analysis. Data are expressed as mean±SD. Significant differences: *P<0.05, **P<0.01 versus WT; $P<0.05, $$P<0.01 versus LLC WT or MC38 WT.

FIG. 3A-C shows the absence of FNDC5/irisin preserves skeletal muscle and function in LLC and MC38 hosts. Gastrocnemius (GSN), quadriceps and tibialis anterior muscle mass expressed in grams (FIG. 3A). In vivo muscle contractility (plantarflexion torque) expressed as a fold change versus WT (FIG. 3B). Total locomotor activity represented as ambulatory, stereotypic movement and resting time expressed in seconds, distance expressed in inch and speed expressed in inch/seconds (FIG. 3C). Data are expressed as mean±SD. Significant differences: *P<0.05, **P<0.01, ***P<0.001 versus WT; $P<0.05, $$P<0.01, $$$P<0.001 versus LLC WT or MC38 WT.

FIG. 4A-D shows the absence of FNDC5/irisin normalizes markers of protein catabolismuantification and representative Western Blot for pSTAT3/STAT3 in whole quadriceps muscle (FIG. 4A). Gene expression levels in the quadriceps muscle for Atrogin and Murf1 measured by quantitative PCR and normalized to TBP levels (FIG. 4B). Schematic representation of the ex vivo experiment. Muscle precursors (SCs) were isolated from WT and KO muscle, differentiated into myotubes and exposed by means of trans wells to LLC tumor cells (FIG. 4C). Quantification and representative Western Blot for pSTAT3/STAT3 in WT and KO primary myotubes exposed to LLC tumor cells for 48 hours (FIG. 4D). Data are expressed as mean±SD. Significant differences: *P<0.05, **P<0.01, ***P<0.001 versus WT; $$P<0.01, $$$P<0.001 versus LLC WT.

FIG. 5A-C shows the absence of FNDC5/irisin normalizes skeletal muscle metabolism. Gene expression levels in the quadriceps muscle for PDK4 measured by quantitative PCR and normalized to TBP levels (FIG. 5A). Representative Western blotting and quantification (expressed as fold change versus WT) for PDK4 (blot 2; tubulin used as loading control, FIG. 5B).

The enzymatic activities of succinate dehydrogenase (SDH) in the skeletal muscle were expressed in milliunits/μL (nmole/min/μl, FIG. 5C). Data are expressed as mean±SD. Significant differences: *P<0.05, **P<0.01 versus WT; $P<0.05, $$P<0.01 versus LLC WT.

FIG. 6 shows tissue weights in LLC and MC38 hosts. Heart, liver, white adipose tissue (WAT) and spleen mass expressed in grams. Data are expressed as mean±SD. Significant differences: *P<0.05, **P<0.01 versus WT; $$$P<0.05 versus LLC WT.

FIG. 7 shows the absence of FNDC5/irisin preserves trabecular bone mass in LLC hosts. Assessment of trabecular bone volume fraction (BV/TV; expressed as %), trabecular thickness (Tb.Th; expressed as μm), trabecular separation (Tb.Sp; expressed as μm), trabecular number (Tb.N; expressed as 1/μm), trabecular pattern factor (Tb.Pf, expressed as 1/μm) and connectivity density (Conn.Dn; expressed as 1/μm3) in the femurs from the same mice. Data are expressed as mean±SD. Significant differences: *P<0.05, *P<0.01 vs. WT; $P<0.05, $$P<0.01, $$$P<0.001 versus LLC WT.

FIG. 8 shows that absence of FNDC5/irisin increases bone size. Three-D rendering, and assessment cortical bone volume fraction (BV/TV; expressed as %), cross-sectional thickness (Cs·Th; expressed in mm), endosteal perimeter (expressed in mm) and medullary area (expressed in mm3). Data are expressed as mean±SD. Significance of the differences: *P<0.05, *P<0.01vs. WT; $P<0.05, $$P<0.01, $$$P<0.001 versus LLC WT.

### DETAILED DESCRIPTION

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers ±10% thereof, or 10% below the lower listed limit and 10% above the higher listed limit for the values listed for a range.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human. FNDC5/Irisin Inhibitors

The current invention contemplates that FNDC5/irisin can be neutralized or inhibited by several different non-limiting methods. For example, as described herein, FNDC5/irisin can be neutralized or inhibited by administration of a therapeutically effective amount of an agent where the agent comprises an adeno-associated virus (AAV) or lentovirus-containing an a short-hairpin RNA (shRNA) against FNDC5/irisin (shFNDC5/irisin). In some embodiments, the shFNDC5/irisin is commercially available and can be attached to or part of any vector known in the art including plasmids, viral vectors, bacteriophages, cosmids, and artificial chromosomes.

Alternatively, as described herein, FNDC5/irisin can be neutralized or inhibited by administration of a therapeutically effective amount of an agent where the agent comprises an antibody, bivalent antibody or a monoclonal antibody directed against the FNDC5/irisin.

Further, as described herein, FNDC5/irisin can be neutralized or inhibited by administration of a therapeutically effective amount of an agent where the agent comprises an siRNA or antisense oligonucleotide that targets FNDC5/irisin.

Also, as contemplated herein, FNDC5/irisin can be neutralized or inhibited by administration of a therapeutically effective amount of an agent where the agent comprises an antagonist that binds to a FNDC/irisin-mediated receptor and prevents the binding of FNCS/irisin.

The FNDC/irisin inhibitor or inhibitors or a composition therein can be administered once per day, two or more times daily or once per week. The FNDC/irisin inhibitor or inhibitors or composition containing the same can occur by any conventional means including orally intramuscularly, intraperitoneally or intravenously into the subject. If injected, they can be injected at a single site per dose or multiple sites per dose.

### FNDC5/Irisin Antibodies and Related Inhibitors

More specifically an FNDC5/irisin inhibitor is an antibody directed against an FNDC5/irisin. The FNDC5/irisin antibody can also include an antibody fragment or a bivalent antibody or fragment thereof, inhibiting FNDC5/irisin. As described herein, the FNDC5/irisin inhibitor may be part of a pharmaceutical composition where the composition may include either an antibody or fragment thereof for FNDC5/irisin.

The FNDC5/irisin antibodies described herein can be made or obtained by any means known in the art, including commercially. It is also contemplated that an antibody can be specifically reactive with an FNDC5/irisin polypeptide may also be used as an antagonist. An anti-FNDC5/irisin antibody herein may be an antibody or fragment thereof that binds to FNDC5/irisin.

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” and other interchangeable terms for similar binding fragments such as described below.

Native antibodies and native immunoglobulins are usually heterotetrameric glycoproteins of about 150,000 Daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}” or “VH”) followed by a number of constant domains (“C_{H}” or “CH”). Each light chain has a variable domain at one end (“V_{L}” or “VL”) and a constant domain (“C_{L}” or “CL”) at its other end; the constant domain of the light chain is aligned with the first constant domain of the heavy chain, and the light-chain variable domain is aligned with the variable domain of the heavy chain. Particular amino acid residues are believed to form an interface between the light-and heavy-chain variable domains.

The FNDC5/irisin inhibitors as described herein can be a “synthetic polypeptide” derived from a “synthetic polynucleotide” derived from a “synthetic gene,” meaning that the corresponding polynucleotide sequence or portion thereof, or amino acid sequence or portion thereof, is derived, from a sequence that has been designed, or synthesized de novo, or modified, compared to an equivalent naturally occurring sequence. Synthetic polynucleotides (antibodies or antigen binding fragments) or synthetic genes can be prepared by methods known in the art, including but not limited to, the chemical synthesis of nucleic acid or amino acid sequences. Synthetic genes are typically different from naturally occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

With respect to FNDC5/irisin antibodies, the term “antigen” refers to the proteins FNDC5/irisin, respectively or any fragment of the protein molecules thereof.

The terms “antigen-binding portion of an antibody,” “antigen-binding fragment,” “antigen-binding domain,” “antibody fragment” or a “functional fragment of an antibody” are used interchangeably herein to refer to one or more fragments of an antibody that retain the ability to specifically bind to FNDC5/irisin.

It is contemplated that the FNDC5/irisin antibodies may also include “diabodies” which refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. See for example, EP 404,097; WO 93/11161; and Hollinger et al., Proc. Natl. Acad. Sci. USA 90:6444 6448 (1993).

It is contemplated that the FNDC5/irisin antibodies may also include “chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin are inserted in place of the murine Fc. See for example, Jones et al., Nature 321:522-525 (1986); Reichmann et al., Nature 332:323-329 (1988); and Presta, Curr. Op. Struct. Biol., 2:593-596 (1992).

It is contemplated that the FNDC5/irisin antibodies may also include a “monoclonal antibody” which refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which can include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, monoclonal antibodies can be made by a hybridoma method, recombinant DNA methods, or isolated from phage antibody.

As used herein, “immunoreactive” refers to binding agents, antibodies or fragments thereof that are specific to a sequence of amino acid residues on the FNDC5/irisin protein (“binding site” or “epitope”), yet if are cross-reactive to other peptides/proteins, are not toxic at the levels at which they are formulated for administration to human use. The term “binding” refers to a direct association between two molecules, due to, for example, covalent, electrostatic, hydrophobic, and ionic and/or hydrogen-bond interactions under physiological conditions and including interactions such as salt bridges and water bridges and any other conventional binding means. The term “preferentially binds” means that the binding agent binds to the binding site with greater affinity than it binds unrelated amino acid sequences.

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1femtomolar (fM). As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

“Epitope” refers to that portion of an antigen or other macromolecule capable of forming a binding interaction with the variable region binding pocket of an antibody.

The term “specific” refers to a situation in which an antibody will not show any significant binding to molecules other than the antigen containing the epitope recognized by the antibody. The term is also applicable where, for example, an antigen binding domain is specific for a particular epitope which is carried by a number of antigens, in which case the antibody will be able to bind to the various antigens carrying the epitope. The terms “preferentially binds” or “specifically binds” mean that the antibodies bind to an epitope with greater affinity than it binds unrelated amino acid sequences, and, if cross-reactive to other polypeptides containing the epitope, are not toxic at the levels at which they are formulated for administration to human use.

The term “binding” refers to a direct association between two molecules, due to, for example, covalent, electrostatic, hydrophobic, and ionic and/or hydrogen-bond interactions under physiological conditions and includes interactions such as salt bridges and water bridges, as well as any other conventional means of binding.

As contemplated herein, an FNDC5/irisin inhibitor may be generated through gene expression technology. The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

### Formulations

Formulations provided herein may include “pharmaceutical compositions,” in addition to an FNDC5/irisin antibody, FNDC5/irisin antibodies or antibody fragments thereof, a pharmaceutically acceptable excipient, carrier, buffer, stabilizer, surfactant or other materials well known to those in the art. Such materials should be non-toxic and should not interfere with the efficacy of the active ingredient(s). The precise nature of the carrier or other material will depend on the route of administration.

It is contemplated that any formulation of FNDC5/irisin antibodies or other inhibitors can include pre-filled syringes containing the formulations, and the use of such formulations useful for treating any disorder described herein.

In one aspect, the formulation is stable following preparation, which can be tested according to conventional means. Safe handling and administration of formulations comprising proteins represent significant challenges to pharmaceutical formulators. Proteins possess unique chemical and physical properties that present stability problems: a variety of degradation pathways exist for proteins, implicating both chemical and physical instability. Chemical instability includes deamination, aggregation, clipping of the peptide backbone, and oxidation of methionine residues. Physical instability encompasses many phenomena, including, for example, aggregation.

A “stable” formulation is one in which the protein therein essentially retains its physical stability and/or chemical stability and/or biological activity upon storage. Various analytical techniques for measuring protein stability are available in the art. See for example “Peptide and Protein Drug Delivery,” pp. 247-301, Vincent Lee, ed., Marcel Dekker, Inc., New York, N.Y., Pubs. (1991).

Formulations as described herein may contain a buffering agent such as, for example, histidine, acetate, citrate or phosphate. Buffering agents may be included in an amount of about 5mM to about 100 mM. In one embodiment, the formulation comprises about 5 mM, about 7.5 mM, about 10 mM, about 12.5 mM, about 15 mM, about 17.5 mM, 20 mM, about 22.5 mM, about 25mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, or any integer therein histidine, acetate, citrate or phosphate. As used herein, when referring to buffer concentrations, the term “about” means±2% of the indicated value.

Formulations may be prepared for any type of administration known for antibodies or other inhibitors as described in more detail below.

Formulations provided herein may further include an acceptable carrier or excipient including any carrier or excipient that is a pharmaceutically acceptable carrier or excipient and which is acceptable for administration to a patient as described in in more detail herein.

In one embodiment, a formulation provided herein is isotonic. Representative isotonic formulations include, but are not limited to, those that are from about 250 to about 350 milliosmolar. In another embodiment, a formulation provided herein is hypertonic. Representative hypertonic formulations include, but are not limited to, those that are from about 351 to about 1000 milliosmolar.

Polyols may be added to a formulation described herein in an amount of up to about 1 M. For example, the formulation may comprise polyol in an amount of about 50 mM, about 75 mM, about 100 mM, about 150 mM, about 200 mM, about 225 mM, about 240 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, about 1 M, or any integer therein. In one embodiment, a formulation provided herein contains polyol in an amount of less than 300 mM and the formulation is made isotonic with a salt in a concentration of from about 100 mM to about 175 mM. For example, the formulation containing polyol in an amount of less than 300 mM is made isotonic with a salt in a concentration of about 130 mM. As used herein, when referring to polyol concentrations, the term “about” means±2% of the indicated value. In one aspect, a polyol to be used in the formulations provided herein may be a sugar such as, for example, a non-reducing sugar. Representative examples of non-reducing sugars include, but are not limited to, trehalose and sucrose. For example, a formulation may comprise from about 200 mM to about 300 mM trehalose or sucrose. In one embodiment, a formulation may comprise about 240 mM trehalose or sucrose. Alternatively, the sugar may be sorbitol in an amount (concentration) of from about 200 mM to about 300 mM. In one embodiment, a formulation may comprise about 240 mM sorbitol.

A formulation provided herein may have any acceptable pharmaceutically acceptable pH of about 4.0 to about 7.5.

One would understand that formulations comprising an antibody or antigen-binding fragment, identified by the methods described herein can be prepared for storage by mixing the protein having the desired degree of purity with optional physiologically acceptable carriers, excipients and/or stabilizers in the form of aqueous solutions.

Acceptable carriers are physiologically acceptable to the administered patient and retain the therapeutic properties of the compounds with/in which it is administered. Acceptable carriers and their formulations are and generally described in, for example, Remington' Pharmaceutical Sciences (18th Edition, ed. A. Gennaro, Mack Publishing Co., Easton, Pa. 1990).

One exemplary carrier is physiological saline. The phrase “pharmaceutically acceptable carrier” as used herein means an acceptable material, composition or vehicle, such as a liquid or solid filler, diluent, excipient, and/or solvent involved in carrying or transporting the subject compounds from the administration site of one organ, or portion of the body, to another organ, or portion of the body. Each carrier is acceptable in the sense of being compatible with the other ingredients of the formulation and not injurious to a subject to whom it is administered. Nor should an acceptable carrier alter the specific activity of the subject compounds.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with administration. Compositions or formulations, therefore, refer to a composition suitable for therapeutic and/or diagnostic use in a subject. Compositions and formulations include an amount of a compound described herein and a pharmaceutically or physiologically acceptable carrier.

Compositions can be formulated to be compatible with a particular route of administration (i.e., systemic or local). Thus, compositions include carriers, diluents, or excipients suitable for administration by various routes.

Compositions can be administered, for example, by injection, including, but not limited to, subcutaneous, intradermal, intravenous, intra-arterial, intraperitoneal, or intramuscular injection. Isotonic agents, for example, sugars, polyalcohols such as manitol, sorbitol, and sodium chloride may be included in the composition. The resulting solutions can be packaged for use as is or lyophilized; the lyophilized preparation can later be combined with a sterile solution prior to administration. For intravenous, injection, or injection at the site of affliction, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's Injection, Lactated Ringer's Injection. Preservatives, stabilizers, buffers, antioxidants and/or other additives may be included, as needed. Sterile injectable solutions can be prepared by incorporating an active ingredient in the required amount in an appropriate solvent with one or a combination of ingredients enumerated above, as required, followed by filtered sterilization.

Any formulation can optionally include one or more surfactants, such as, for example, polysorbate 20 or 80, TWEEN, PLURONIC, F68, or polyethylene glycol (PEG).

When the FNDC5/irisin compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available packages.

The phrase “pharmaceutically acceptable” refers to molecular entities and compositions that are physiologically tolerable and do not typically produce an allergic or similar untoward reaction, such as gastric upset, dizziness and the like, when administered to a subject.

The term “unit dose” when used in reference to a therapeutic composition refers to physically distinct units suitable as unitary dosage for subjects, each unit containing a predetermined quantity of active material calculated to produce the desired therapeutic effect in association with the required diluent, i.e., carrier, or vehicle.

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable but are typified by an initial administration followed by repeated doses at one or more hour-intervals by a subsequent injection or other administration. Alternatively, continuous intravenous infusions sufficient to maintain concentrations in the blood are contemplated.

One embodiment contemplates the use of the compositions described herein to make a medicament for treating a condition, disease or disorder described herein. Medicaments can be formulated based on the physical characteristics of the patient/subject needing treatment and can be formulated in single or multiple formulations based on the stage of the condition, disease or disorder. Medicaments can be packaged in a suitable package with appropriate labels for the distribution to hospitals and clinics wherein the label is for the indication of treating a subject having a disease described herein. Medicaments can be packaged as a single or multiple units. Instructions for the dosage and administration of the compositions can be included with the packages as described below.

Also provided herein is a pre-filled syringe suitable for intravenous or intraperitoneal administration, comprising a formulation described herein. Such pre-filled syringes may be packaged and labeled for use for treatment of an angiogenesis-related condition such as any of the conditions described herein. Packages may further include directions for storage and administration. Provided herein is a package containing one or more pre-filled syringes suitable for intravenous or intravitreal administration comprising the formulation of any of the preceding claims.

### Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

### Effective or Therapeutic Amount

Effective or therapeutic amounts of the compositions of this disclosure include any amount sufficient to inhibit (e.g., slow or stop) the progression of a neurodegenerative disorder. In some embodiments, effective amounts of the compositions include any amount sufficient to inhibit (e.g., slow or stop) the deterioration of the muscular function of a patient.

The amount of the active ingredient that may be combined with the optional carrier materials to produce a single dosage form may vary depending upon the host treated and the particular mode of administration. The specific dose level for any particular patient may depend upon a variety of factors, including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

### Other Definitions

As used herein, “prevention” refers to prophylaxis, prevention of onset of, or symptoms, prevention of a skeletal muscle or other disorder. The methods of the invention comprise administering an agent that inhibits FNDC5/irisin, i.e., an FNDC5/irisin inhibitor. As used herein, “inhibition,” “treatment” and “treating” are used interchangeably.

A “subject” or “patient” (e.g., a mammal such as a human or a non-human animal such as a primate, rodent, cow, horse, pig, sheep, camel, llama, etc.) can be a mammal who exhibits one or more clinical manifestations and/or signs or symptoms of a disease or disorder described herein. In certain situations, a subject may be asymptomatic and yet still have clinical manifestations of the disease or disorder.

### Disorders to be Treated

The present disclosure is based on the discovery that FNDC5/Irisin is an important mediator of skeletal muscle wasting and weakness and bone loss due to cancer and show that FNDC5/Irisin is an important target in the treatment and prevention of cancer cachexia.

The findings shown herein provide evidence that FNDC5/irisin is an important mediator of skeletal muscle wasting and weakness and show that FNDC5/irisin is an important target in the treatment and prevention of cancer cachexia. Thus, neutralizing, blocking, reducing FNDC5/irisin through neutralizing antibody, small molecule inhibitors, shRNA, etc. will reduce or prevent muscle wasting and bone loss with cancer cachexia. Neutralizing antibodies are also provided to irisin in order to block or prevent cancer cachexia.

Methods are provided for treating or preventing muscle wasting and bone loss in cancer cachexia and other disorders. In particular embodiments, the methods involve providing treatments that inhibit or ablate FNDC5/irisin in a particular tumor. Methods of the current disclosure also provide means of preserving body wasting and muscle and bone mass during cancer cachexia and in other conditions associated with skeletal muscle derangements. Methods of targeting FNDC5/irisin are used to prevent other conditions of muscle loss such as sarcopenia with aging, muscle and bone loss with immobilization, and other conditions of muscle loss or wasting.

Further reference is made to the following experimental examples.

### EXAMPLES

The following examples are given for the purpose of illustrating various embodiments of the disclosure and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### General Methods

### Cell culture

Murine Lewis Lung Carcinoma cells (LLC) were provided by Dr. Paola Costelli (University of Torino, Italy) the murine MC38 cells were provided by Dr. Xiongbin Lu (Indiana University, Indianapolis) and both were cultured in DMEM medium supplied with 10% fetal bovine serum, 1% glutamine, 1% sodium pyruvate, and 1% penicillin and streptomycin in a 5% CO2, 37° C. humidified incubator.

### Animals

All animal studies were approved by the Institutional Animal Care and Use Committee at Indiana University School of Medicine and complied with the National Institutes of Health Guidelines for Use and care of Laboratory Animals and with the 1964 Declaration of Helsinki and its later amendments. All animals were maintained on a regular dark-light cycle (light from 8 a.m. to 8 p.m.), with free access to food and water during the whole experimental period. Global Fndc5 KO mice were provided by Dr. Bruce Spiegelman (Harvard University). Briefly, they were generated by crossing FNDC5 floxed mice with Ella-cre mice to generate germline deletion of FNDC5 (Exon 2 and 3). Experiments were performed with age-matched global FNDC5 KO and littermate wild-type control mice. Mice were randomized into 4 groups: control mice WT and KO inoculated with sterile saline and tumor-bearing animals WT and KO inoculated subcutaneously and intrascapularly with 1×10⁶ LLC cells or inoculated intrasplenally with 1.25×10⁵ MC38 cells in sterile saline. The experiments were stopped when the animals reach a stage of severe cancer cachexia. In particular, after 23 days from the LLC injection and 21 days from the MC38 injection. Body composition assessment by dual-energy X-ray absorptiometry

Lean and adipose tissue were assessed by means of dual-energy X-ray absorptiometry (DXA) scanning of formalin-fixed carcasses. According to the manufacturer's guidelines, in order to calibrate and validate the apparatus for its performance, a spine phantom was scanned using the Lunar PIXImus densitometer (PIXImus, Fitchburg, WI, USA) before scanning the first carcass. Animal carcasses were placed in a prone position with the limbs outstretched.

### In Vivo Muscle Contractility

All the animals underwent in vivo plantarflexion torque assessment the day before sacrifice (Aurora Scientific Aurora, ON, Canada). Briefly, the left hind foot was positioned to align with the tibia at 90° and then taped into the footplate force transducer. The knee was clamped at the femoral condyles, and two disposable monopolar electrodes (Natus Neurology, Middleton, WI, USA) were placed subcutaneously posterior/medial to the knee in order to stimulate the tibial nerve. The peak twitch torque was first established in order to determine the maximal stimulus intensity, and mice were exposed to a stimulation of 0.2 ms at 100 Hz to determine the force output.

### Activity Cages

Motor activities in an open field were determined with the Auto-Track Opto-Varimex activity monitoring system (Columbus Instruments, Columbus, OH, USA). The mice were housed in a transparent cage. The system utilizes infra-red beams to calculate animal movement and current position. During the activity measurements, animals had no access to food or chow. All studies were performed under strictly standardized conditions after 7:00 pm for 10 min.

### Western Blotting

Total protein extracts were obtained by lysing cell layers or homogenizing 100 mg quadriceps muscle tissue in radioimmunoprecipitation assay (RIPA) buffer (150 mM NaCl, 1.0% NP-40, 0.5% sodium deoxycholate, 0.1% sodium dodecyl sulfate (SDS), and 50 mM Tris, pH 8.0) completed with protease (Roche, Indianapolis, IN, USA) and phosphatase (Thermo Scientific, Rockford, IL, USA) inhibitor cocktails. Cell debris were removed by centrifugation (15 min, 14000 g), and the supernatant was collected and stored at −80° C. Protein concentration was determined using the bicinchoninic acid (BCA) protein assay method (Thermo Scientific). Protein extracts (30 μg) were then electrophoresed in 4-15% gradient SDS Criterion TGX precast gels (Bio-Rad, Hercules, CA, USA). Gels were transferred to nitrocellulose membranes (Bio-Rad). Membranes were blocked with SEA BLOCK blocking reagent (Thermo Scientific) at room temperature for 1 h, followed by an overnight incubation with diluted antibody in SEA BLOCK buffer containing 0.2% Tween-20 at 4° C. with gentle shaking. After washing with PBS containing 0.2% Tween-20, the membrane was incubated at room temperature for 1 h with either Anti-rabbit IgG (H+L) DyLight 800 or Anti-mouse IgG (H+L) DyLight 600 (Cell Signaling Technologies, Danvers, MA, USA). Blots were then visualized with Odyssey Infrared Imaging System (LI-COR Biosciences, Lincoln, NE, USA). Optical density measurements were taken using the Gel-Pro Analyser software. Antibodies used were PDK4 (ab214938) from Abcam, and a-Tubulin (#12G10) from Developmental Studies Hybridoma Bank.

### Real-time Quantitative Polymerase Chain Reaction

RNA from quadriceps was isolated using the miRNeasy Mini kit (Qiagen, Valencia, CA, USA) and following the protocol provided by the manufacturer. RNA was quantified by using a Synergy H1 spectrophotometer (BioTek, Winooski, VT, USA). RNA integrity was checked by electrophoresis on 1.2% agarose gel containing 0.02 mol/L morpholinopropanesulfonic acid and 18% formaldehyde. Total RNA was reverse transcribed to cDNA by using the Verso cDNA kit (Thermo Fisher Scientific, Waltham, MA, USA). Transcript levels were measured by Real-Time PCR (Light Cycler 96, Roche) taking advantage of the TaqMan gene expression assay system (Life Technologies, Carlsbad, CA, USA). Expression levels for Atrogin-1 (Mm00499523_m1), MuRF-1 (Mm01185221_m1) and PDK4 (Mm01166879_m1) were detected. Gene expression was normalized to TATA-binding protein (TBP) (Mm01277042_m1) levels using the standard 2-ACT methods.

### Succinate Dehydrogenase Enzymatic Activity

The total SDH enzymatic activity was measured using a SDH Colorimetric Assay Kit (#MAK197; Sigma-Aldrich, St. Louis, MO, USA), as per manufacturer's instruction. Briefly, 10 mg of quadriceps muscle tissue were homogenized in 100 μL ice-cold assay buffer and subsequently centrifuged. The supernatant was collected, and the total protein content was measured using the BCA protein assay (Thermo Fisher Scientific, Waltham, MA, USA). Ten microlitres of homogenate were then added to a 96-well plate, along with an appropriate volume of reaction mix, and then incubated at 37° C. The absorbance at 600 nm was measured after 0, 3, and 20 min. The rate of absorbance decrease between 3 and 20 min was corrected for the protein concentration and used to calculate the SDH content.

### Microcomputed Tomography (μCT) Analysis Of Femurs Bone Morphometry

MicroCT (μCT) scanning was performed to measure morphological indices of metaphyseal regions of femurs, as described in Bouxsein et al. After euthanasia, the mouse carcasses were fixed for 2 days in 10% neutral buffered formalin, transferred into 70% ethanol, the right femurs dissected, and prepared for μCT scanning on a high-throughput μCT specimen scanner Bruker Skyscan 1176 (Bruker, Kontich, Belgium). Bone samples were rotated around their long axes and images were acquired with the following parameters: pixel size =9 μm3; peak tube potential=50 kV; X-ray intensity=500 μA; 0.3° rotation step. Raw images were reconstructed using SkyScan reconstruction software (NRecon; Bruker, Kontich, Belgium) to 3-dimensional cross-sectional image data sets using a 3-dimensional cone beam algorithm. Structural indices were calculated on reconstructed images using the Skyscan CT Analyzer software (CTAn; Bruker, Kontich, Belgium). Trabecular bone was separated using a custom processing algorithm in CTAn, based on the different thicknesses of the structures. Trabecular bone was analyzed between 0.5 mm and 1.5 mm for the ES-2 model and between 1 mm and 2 mm for the C26 and LLC models under the femoral distal growth plate using a threshold of 80-255. Trabecular parameters included bone volume fraction (BV/TV), number (Tb·N), thickness (Tb·Th), separation (Tb·Sp), connectivity (Conn.Dn) and pattern factor (Tb·Pf). The Tb·Pf is a measure of trabecular connectedness that describe the three-dimensional configuration of trabeculae. In particular, considering the relation between convex and concave trabecular surfaces indicates if the structures are well-connected or not. Cortical bone was analyzed by threshold of 160-255 in femoral mid-shaft. Cortical parameters included bone volume fraction (BV/TV), cross-sectional thickness (Cs·Th), endosteal perimeter and medullary area.

### Example 1

Role of FNDC5/Irisin on Skeletal Muscle During Cancer-Induced Cachexia

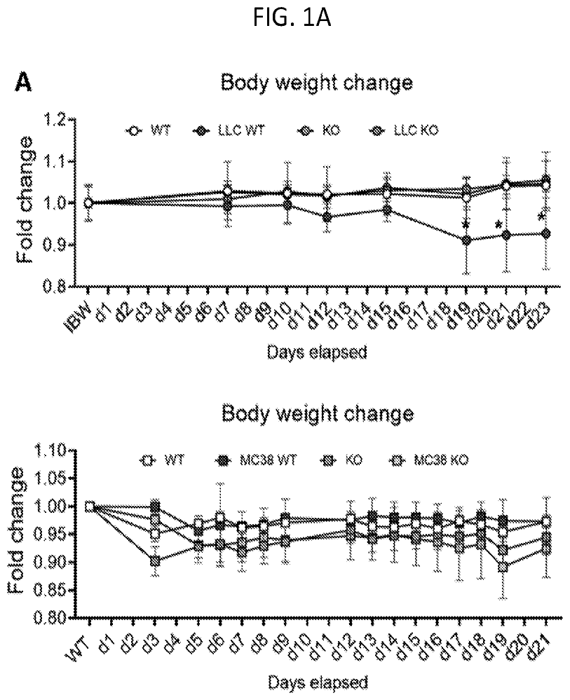

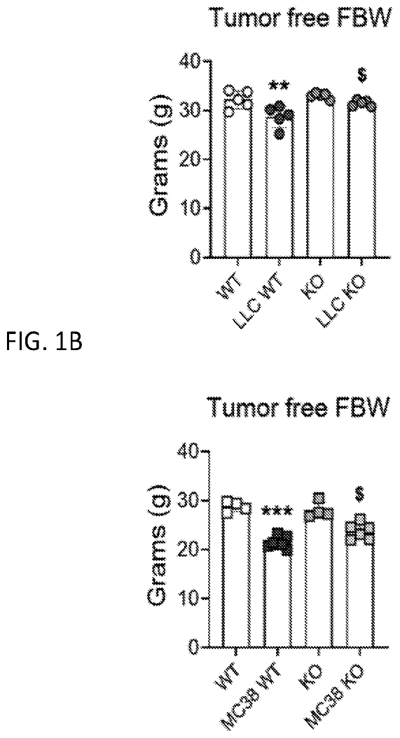

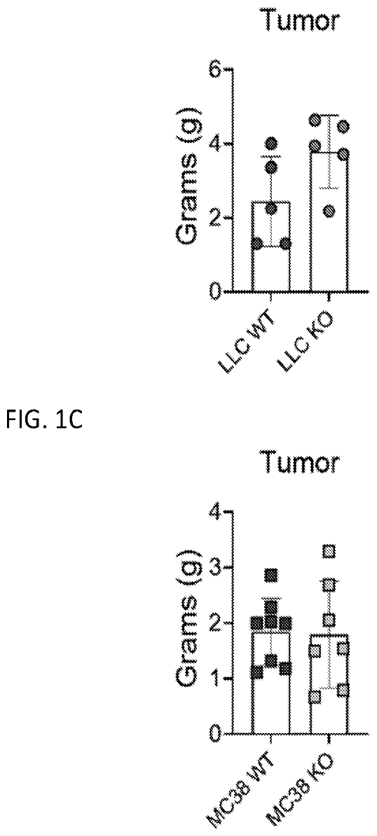

To investigate the role of FNDC5/irisin on skeletal muscle during cancer-induced cachexia, FNDC5/irisin deficient KO mice (n=5) were injected subcutaneously (s.c.) and intrascapularly with 1x106 LLC cells or intra-splenally with 1.25×10⁵ MC38 cells. LLC tumor-bearing mice were sacrificed after 23 days and MC38 bearers after 21 days following tumor transplantation when the control mice reached a severe degree of cachexia (−12% vs. initial body weight FIG. 1A). There was no significant difference in initial body weight and lean mass content as shown by the DEXA analysis (data not shown). Moreover, no differences were observed in the tumor size at the time of the sacrifice between WT and KO (LLC WT: 2.8+1.3 g; LLC KO 3.5+1g, and MC38 WT: 1.8+0.6 g; MC38 KO 1.79+0.9 g, FIG. 1C). The LLC KO (KO mice bearing the LLC tumor) maintained body weight over the course of the experiment, in stark contrast to the LLC WT (WT mice bearing the LLC tumor) which progressively lost weight with increased tumor growth (FIG. 1A and B).

### Example 2

Effects of the FNDC5/Irisin Ablation on Body Composition.

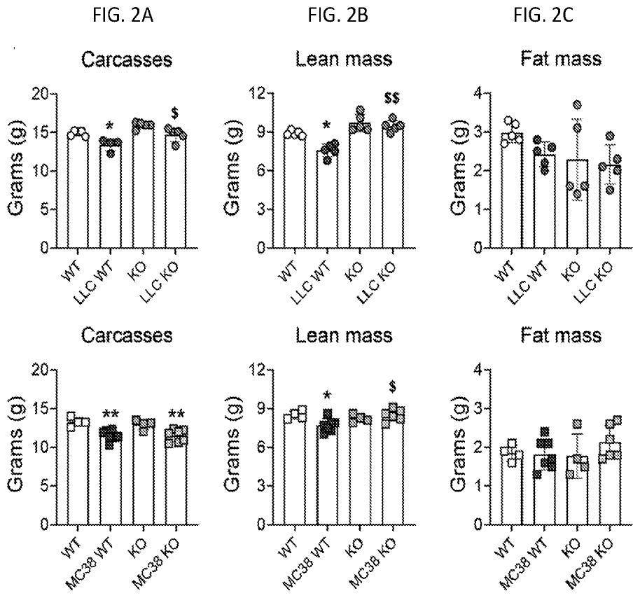

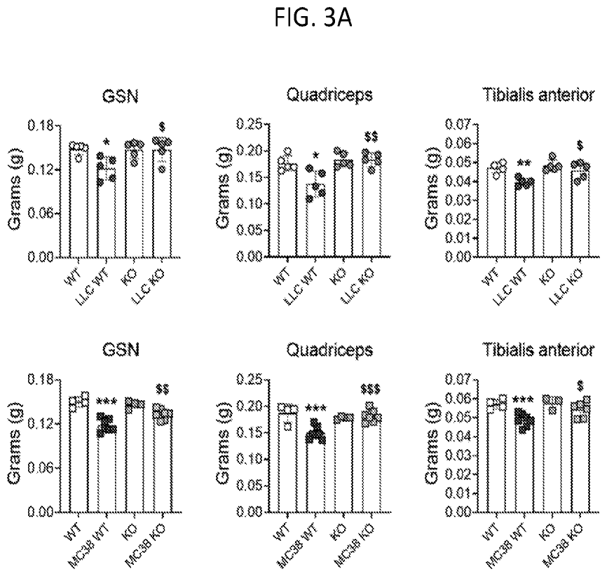

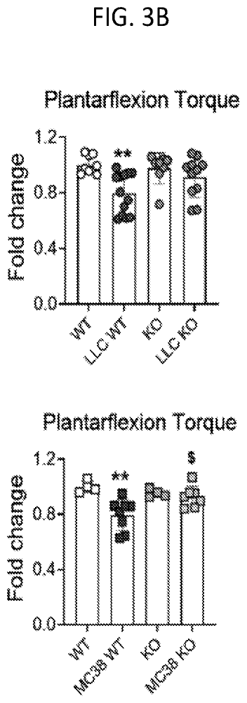

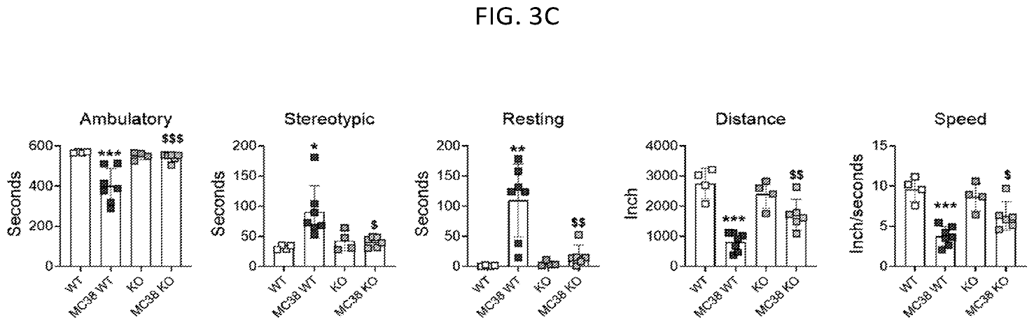

In order to understand the effects of the FNDC5/irisin ablation on body composition, the day before the sacrifice, the animals were analyzed by DXA. The amount of lean tissue was significantly reduced in the LLC and MC38 WT, but the LLC and MC38 KO did not lose any lean mass (FIG. 2B). No differences were observed between the groups with regards to fat mass (FIG. 2C). Carcass weights in tumor hosts (LLC and MC38 WT) showed a significant loss in weight compared with WT animals. In stark contrast, in the LLC KO mice, there was no loss of carcass weight was maintained at same level as control animals (FIG. 2A). Next, the inventors assessed whether the absence of FNDC5 affected skeletal muscle mass. In line with the trend detected in body mass and lean mass, the LLC and MC38 WT lost significant skeletal muscle mass (FIG. 3A), but the LLC and MC38 KO maintained muscle mass comparable with the KO and WT control animals. This was observed in the GSN, quadriceps, Tibialis anterior, EDL, and soleus muscle (FIG. 3A). Both, LLC and MC38 WT bearers experience severe muscle weakness, as showed by the reduction of the in vivo muscle contractility (FIG. 3B). Contrarily, the absence of FNDC5/irisin protected against the reduction of the plantariflexion torque (FIG. 3B). Moreover, as showed by the quantification of the total movements, distance and speed recorded by mean of activity monitoring cages, also the total locomotor activity was preserved in the FNDC/irisin KO mice at level of the control mice (FIG. 3C).

### Example 3

Possible Changes in in Muscle-Specific Signaling Pathways and Muscle Energy Metabolism

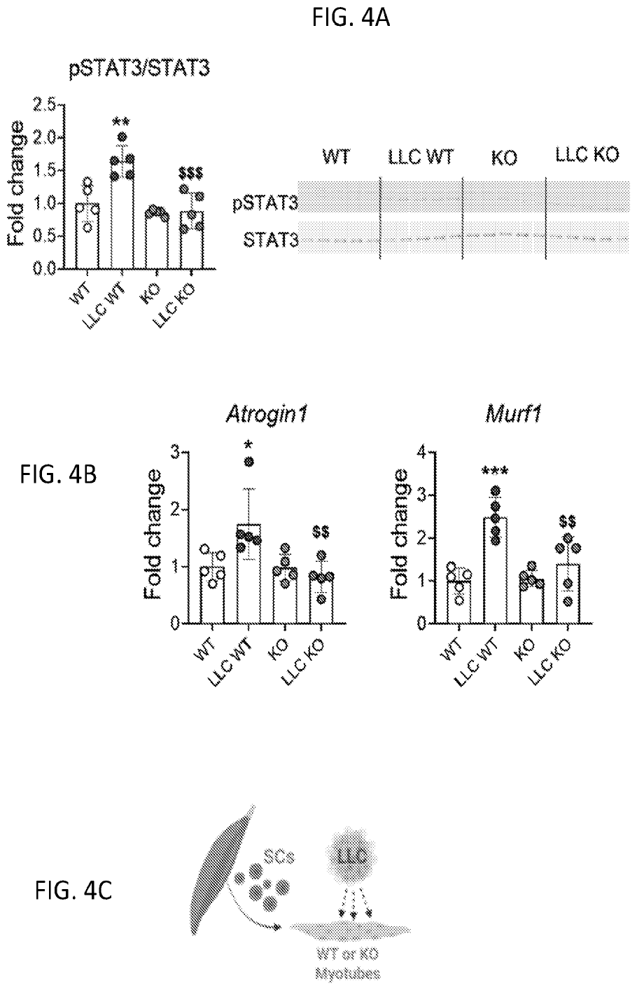

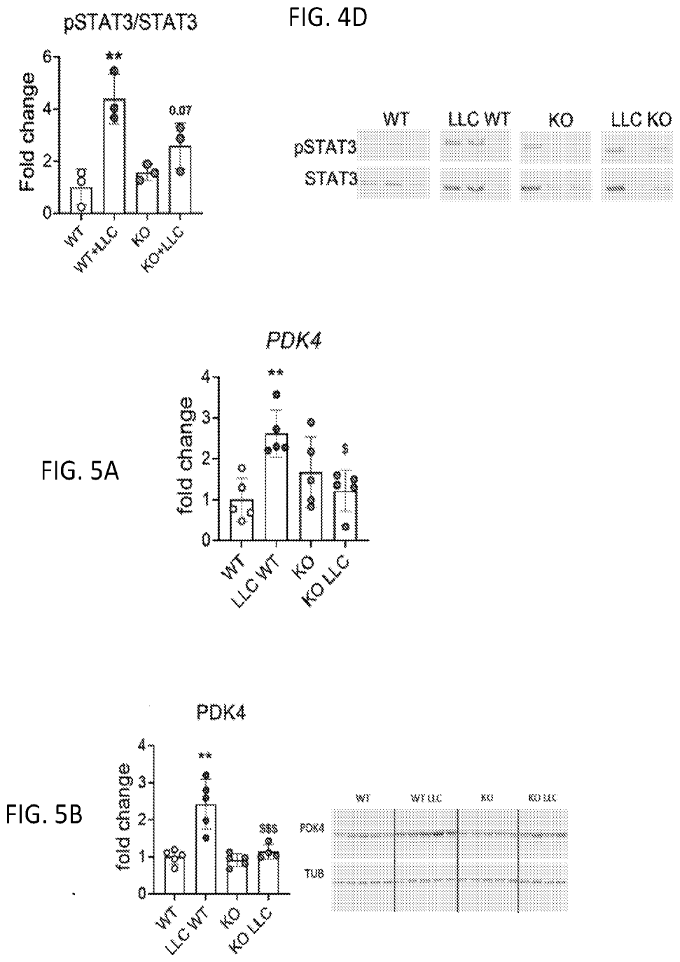

In order to investigate whether the observed muscle phenotype was also associated with changes in muscle-specific signaling pathways, the inventors assessed the levels of protein and genes known to be involved in the regulation of skeletal muscle mass and protein catabolismin particular, the LLC WT showed increased phosphorylation of STAT3 as well as increased levels of Atroginl and Murf1, regulators of muscle catabolismormally overexpressed in conditions associated with muscle atrophy (FIG. 4A and B). The absence of FNDC5/irisin was able to completely prevent the hypercatabolic state normally observed in the muscle of WT mice as shown by the maintenance of normal levels of pSTAT3, Atroginl and Murf1 in the muscle of the LLC KO mice (FIG. 4A and B). Moreover, to better clarify if the absence of FNDC5 is mediated the reduction of the pSTA3/STAT3 ration an ex vivo experiment was performed. Skeletal muscle precursors (SCs) were isolated from WT and KO muscle, cultured, differentiated in myotubes end exposed for 48 hours to LLC cells. Also in vitro the pSTA3/STAT3 ration in increased in the WT myotubes, but was interestingly reduced, also if not significantly in the KO-derived myotubes (FIG. 4C and D).

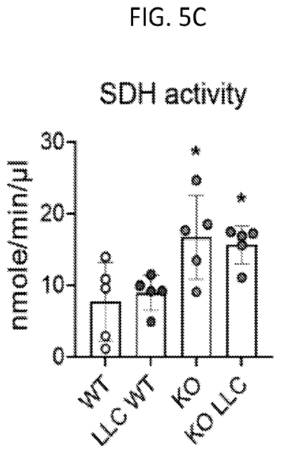

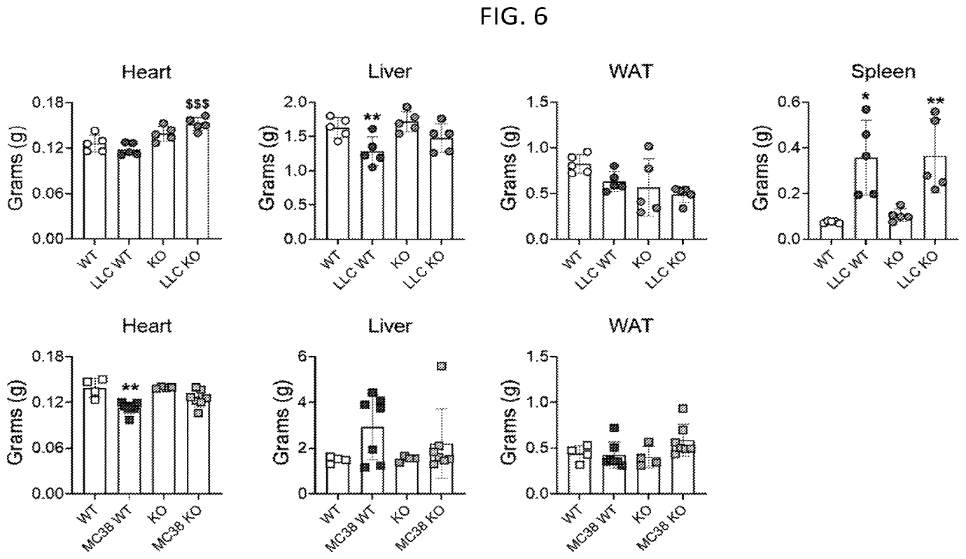

The inventors previously have demonstrated that skeletal muscle wasting induced by cancer is accompanied by depletion of several mitochondrial proteins as well as changes in oxidative metabolism. In order to determine the effect of FNDC5/irisin deletion on muscle energy metabolism, the inventors determined the expression of pyruvate dehydrogenase kinase 4, PDK4, a key enzyme regulating the flow of energy substrates through glycolysis and the TCA cycle. Again, the elevated gene and protein expression of PDK4 observed in the skeletal muscle of the LLC WT was not observed in the skeletal muscle of the LLC KO mice (FIG. 5A and B). The inventors also assessed the enzymatic activity of Succinate dehydrogenase, SDH, an important mitochondrial enzyme. SDH activity was remarkably unchanged in the LLC KO quadriceps muscle compared to LLC WT control animals (FIG. 5C). Notably, the ablation of FNDC5/irisin was able to prevent the loss in heart mass compared with LLC WT (FIG. 6). Whereas a significant reduction of liver mass was observed in the LLC WT, no significant reduction was observed in the LLC KO (FIG. 6). There were no significant differences between LLC WT and LLC KO indicating that FNDC5/irisin ablation did not protect against spleen enlargement. (FIG. 6). There were no significant differences in gonadal adipose tissue (WAT) in this experiment (FIG. 6).

### Example 4

Further Effects of Greater BMC and BMD in the FNDC/Irisin Deficient KO Mice

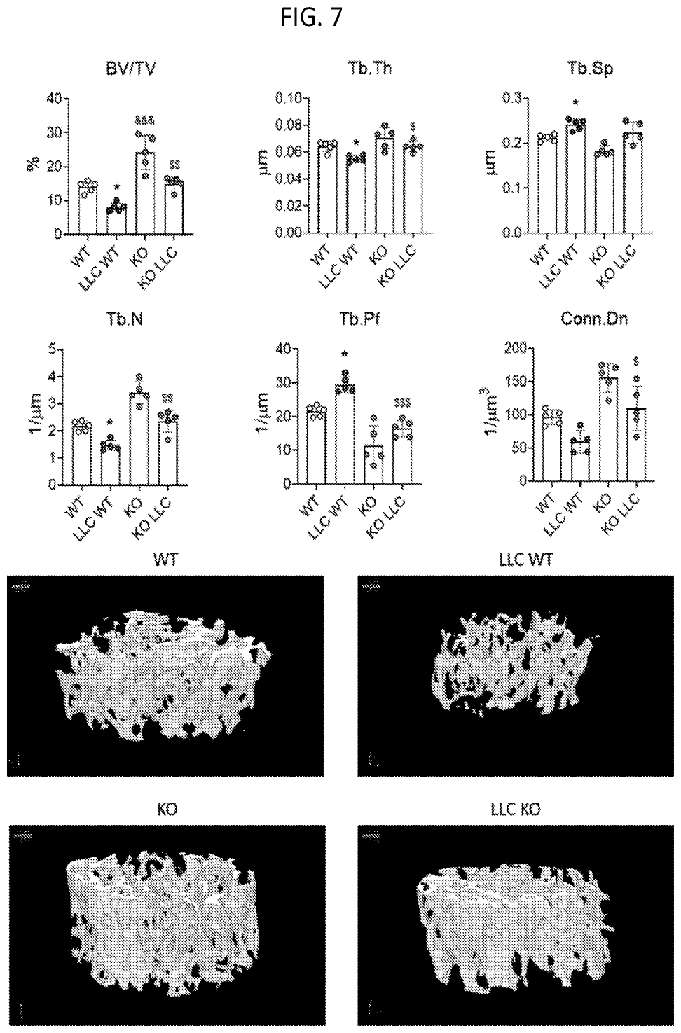

In order to determine whether the greater BMC and BMD in the KO mice was also consistent with increase of bone mass, micro computed tomography, μCT, was performed on femurs from these mice. The LLC WT tumor hosts exhibited a severe loss of femoral trabecular bone (FIG. 7). This was evidenced by markedly reduced trabecular bone volume fraction (BV/TV), reduced trabecular thickness (Tb.Th), trabecular number (Tb.N) and connectivity density (Conn.Dn) in conjunction with increased trabecular separation (Tb.Sp) and pattern factor (Tb.pf) (FIG. 7). As shown by DXA analysis, μCT also showed that the FNDC5/irisin animals had higher bone mass than their WT controls and lost less bone with tumor (FIG. 7). The ablation of FNDC5/irisin significantly preserved the trabecular bone in the tumor-bearing mice, as suggested by greater BV/TV, Tb.Th, Tb.N and Conn.Dn, and by less Tb.Sp and Tb.Pf in the LLC KO than the LLC WT (FIG. 7).

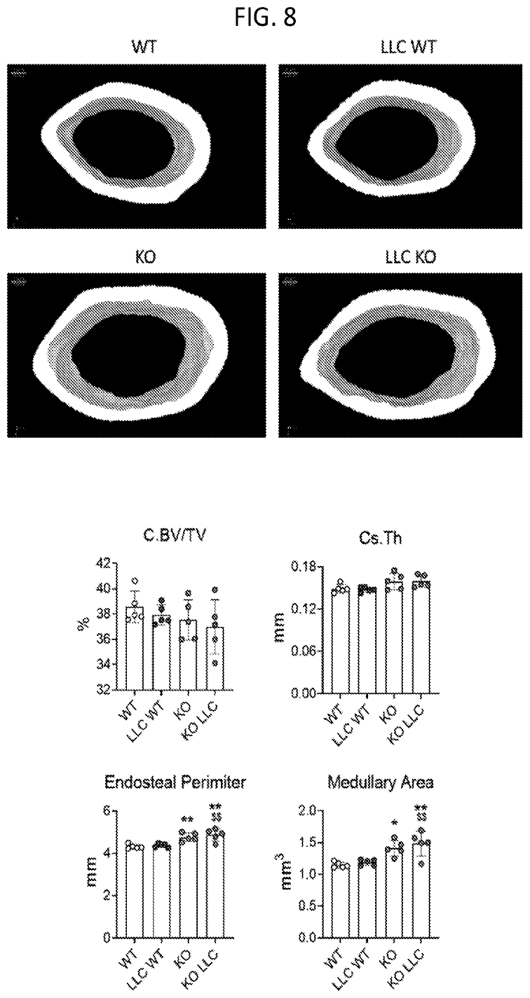

Finally, analysis of the cortical bone volume fraction (BV/TV) and cross-sectional thickness (Cs.Th) did not show significant differences in any group (FIG. 8). However, the endosteal perimeter as well as the medullary area were greater in the KO mice compared to the WT control. Neither WT nor KO mice had any changes in these two parameters due to tumor (FIG. 8).

### Example 5

A further study is to be conducted to elucidate the mechanism of action of inhibiting or depleting FNDC5/irisin. The study determines if FNDC5 is acting as a receptor in muscle or whether another function, such as serving as the precursor to irisin, is responsible. Blocking the expression of FNDC5 has been established to reduce muscle wasting, and the studies analyze whether FNDC5's known function of producing irisin or other function accounts for its advantageous effect. Studies are conducted utilizing a variety of approaches such as specific antibodies, small molecule antagonists, siRNA, miRNA, and CRISPR Cas.

As will be appreciated from the descriptions herein, a wide variety of aspects and embodiments are contemplated by the present disclosure, examples of which include, without limitation, the aspects and embodiments listed below:

Methods for global deletion of FNDC5/irisin in order to demonstrate the potential therapeutic benefits in muscle wasting and fatigue.

Methods for treating or preventing skeletal muscle wasting and weakness through inhibition or depletion of FNDC5/irisin in the treatment and prevention of cancer cachexia.

Methods for neutralizing, blocking, reducing FNDC5/irisin through neutralizing antibody, small molecule inhibitors, shRNA, etc. in order to reduce or prevent muscle wasting with cancer cachexia.

Methods of providing a neutralizing antibody to FNDC/irisin to block cancer cachexia.

Methods and compositions for treating or preventing muscle wasting in cancer cachexia and other disorders.

Methods for providing treatments that inhibit or ablate FNDC5/irisin in a particular tumor.

Methods of inhibiting body wasting and preserving muscle mass during cancer cachexia and in other conditions associated with skeletal muscle derangements.

Methods of targeting FNDC5/irisin are used to prevent other conditions of muscle loss such as sarcopenia with aging, muscle loss with immobilization, and other conditions of muscle loss.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating or preventing muscle wasting in a patient in need thereof comprising administration of a therapeutically effective amount of a pharmaceutical composition comprising an FNDC5/irisin inhibitor wherein composition neutralizes, blocks, or reduces FNDC5/irisin.

2. The method of claim 1, wherein the FNDC5/irisin inhibitor is selected from the group consisting of one or more of an antibody or antibody fragment, a vector containing a short-hairpin RNA (shRNA) against FNDC5/irisin (shFNDC5/irisin), or an antagonist of an FNDC5/irisin receptor.

3. The method of claim 2, wherein the vector is selected from the group consisting of plasmids, viral vectors, bacteriophages, cosmids, and artificial chromosomes.

4. The method of claim 3, wherein the viral vector comprises an adeno-associated virus (AAV) or a lentivirus.

5. The method of claim 2, wherein the FNDC5/irisin inhibitor comprises a monoclonal antibody directed against FNDC5/irisin.

6. The method of claim 1, wherein the FNDC5/irisin inhibitor comprises an siRNA targeting FNDC5/irisin inhibitor.

7. A method for treating or preventing bone loss in a patient in need thereof comprising administration of a therapeutically effective amount of a pharmaceutical composition comprising an FNDC5/irisin inhibitor wherein composition neutralizes, blocks, or reduces FNDC5/irisin.

8. The method of claim 7, wherein the FNDC5/irisin inhibitor is selected from the group consisting of one or more of an antibody or antibody fragment, a vector containing a short-hairpin RNA (shRNA) against FNDC5/irisin (shFNDC5/irisin), or an antagonist of an FNDC5/irisin receptor.

9. The method of claim 8, wherein the vector is selected from the group consisting of plasmids, viral vectors, bacteriophages, cosmids, and artificial chromosomes.

10. The method of claim 9, wherein the viral vector comprises an adeno-associated virus (AAV) or a lentivirus.

11. The method of claim 8, wherein the FNDC5/irisin inhibitor comprises a monoclonal antibody directed against FNDC5/irisin.

12. The method of claim 7, wherein the FNDC5/irisin inhibitor comprises an siRNA targeting FNDC5/irisin inhibitor.

13. The method of claim 1, wherein the pharmaceutical composition depletes skeletal muscle of FNDC5/irisin.

14. The method of claim 7, wherein the pharmaceutical composition depletes skeletal muscle of FNDC5/irisin.

15. A method for treating or preventing treating cancer cachexia in a patient in need thereof comprising administration of a therapeutically effective amount of a pharmaceutical composition comprising an FNDC5/irisin inhibitor wherein composition neutralizes, blocks, or reduces FNDC5/irisin.

16. The method of claim 15, wherein the FNDC5/irisin inhibitor is selected from the group consisting of one or more of an antibody or antibody fragment, a vector containing a short-hairpin RNA (shRNA) against FNDC5/irisin (shFNDC5/irisin), or an antagonist of an FNDC5/irisin receptor.

17. The method of claim 16, wherein the vector is selected from the group consisting of plasmids, viral vectors, bacteriophages, cosmids, and artificial chromosomes.

18. The method of claim 17, wherein the viral vector comprises an adeno-associated virus (AAV) or a lentivirus.

19. The method of claim 16, wherein the FNDC5/irisin inhibitor comprises a monoclonal antibody directed against FNDC5/irisin.

20. The method of claim 15, wherein the FNDC5/irisin inhibitor comprises an siRNA targeting FNDC5/irisin inhibitor.

21. The method of claim 15, wherein the pharmaceutical composition depletes skeletal muscle of FNDC5/irisin.

22. The method of claim 1, wherein the patient in need thereof has been diagnosed with condition of muscle loss or wasting, wherein the condition is selected from the group consisting of cancer, sarcopenia with aging, and chronic immobilization.

23. A pharmaceutical composition comprising an FNDC5/irisin inhibitor administered to a patient in need thereof, wherein administration of the pharmaceutical composition treats or prevents muscle wasting.

24. The pharmaceutical composition of claim 23, wherein the composition neutralizes, blocks, or reduces FNDC5/irisin.

25. The pharmaceutical composition of claim 23, wherein the pharmaceutical composition depletes skeletal muscle of FNDC5/irisin.

26. The pharmaceutical composition of claim 23, wherein the patient in need thereof has been diagnosed with condition of muscle loss or wasting, wherein the condition is selected from the group consisting of cancer, cancer cachexia, sarcopenia with aging, and chronic immobilization.

27. The pharmaceutical composition of claim 23, wherein the pharmaceutical composition further prevents bone loss.

28. The pharmaceutical composition of claim 23, wherein the FNDC5/irisin inhibitor is selected from the group consisting of one or more of an antibody or antibody fragment, a vector containing a short-hairpin RNA (shRNA) against FNDC5/irisin (shFNDC5/irisin), or an antagonist of an FNDC5/irisin receptor.

29. The pharmaceutical composition of claim 28, wherein the vector is selected from the group consisting of plasmids, viral vectors, bacteriophages, cosmids, and artificial chromosomes.

30. The pharmaceutical composition of claim 28, wherein the FNDC5/irisin inhibitor comprises a monoclonal antibody directed against FNDC5/irisin or an siRNA targeting FNDC5/irisin inhibitor.

31. The pharmaceutical composition of claim 23, wherein the pharmaceutical composition can further comprise one or more pharmaceutically acceptable carriers, diluents or excipients and optionally one or more additional therapeutic agents.

32. The method of claim 1, wherein the method further comprises the administration of one or more additional therapeutic agents.
